SEQUENCE LISTING (28 sequences)

SEQ ID NO: 1 - DNA, 27 bp, Artificial
  PCR primer
gaaaacacagtgtgacatggagggctg

SEQ ID NO: 2 - DNA, 24 bp, Artificial
  PCR primer
aactcgtgagaacggtgaatgaag

SEQ ID NO: 3 - DNA, 25 bp, Artificial
  PCR primer
ggtgcctctgtcaacaaaggtgatg

SEQ ID NO: 4 - DNA, 24 bp, Artificial
  PCR primer
atccctttcaaccatgctgacacc

SEQ ID NO: 5 - DNA, 24 bp, Artificial
  PCR primer
tcccccttgcatccctaatcatgc

SEQ ID NO: 6 - DNA, 25 bp, Artificial
  PCR primer
tgagaggaggaagttacagcacagc

SEQ ID NO: 7 - DNA, 25 bp, Artificial
  PCR primer
gcaatcagagggctgaaacacttgg

SEQ ID NO: 8 - DNA, 25 bp, Artificial
  PCR primer
aaaaggctgtaaagatcaaaccacc

SEQ ID NO: 9 - DNA, 26 bp, Artificial
  PCR primer
tcatatctctggacatatctgtcagg

SEQ ID NO: 10 - DNA, 25 bp, Artificial
  PCR primer
tagttcttgggaagctctgcaaagc

SEQ ID NO: 11 - DNA, 26 bp, Artificial
  PCR primer
agtaatcagatttgttctataggtcc

SEQ ID NO: 12 - DNA, 24 bp, Artificial
  PCR primer
gggaatagctatacaattgtataa

SEQ ID NO: 13 - DNA, 26 bp, Artificial
  PCR primer
gctggttttatcagggggattcttgg

SEQ ID NO: 14 - DNA, 27 bp, Artificial
  PCR primer
ttaagtaaatatcaaggggagtttggg

SEQ ID NO: 15 - DNA, 25 bp, Artificial
  PCR primer
cagtaggggatggatgctaacatga

SEQ ID NO: 16 - DNA, 24 bp, Artificial
  PCR primer
agtccacagatcctaccactgctg

SEQ ID NO: 17 - DNA, 16 bp, Artificial
  PCR primer
tccctgggagactcac

SEQ ID NO: 18 - DNA, 25 bp, Artificial
  PCR primer
tctggctcctatcggctagcagaag

SEQ ID NO: 19 - DNA, 20 bp, Artificial
  PCR primer
ggatggtatcgcttattcct

SEQ ID NO: 20 - DNA, 28 bp, Artificial
  PCR primer
tcgcacagtggaggaaacttctagtcct

SEQ ID NO: 21 - DNA, 24 bp, Artificial
  PCR primer
ctcagcagacctcagtccatcacc

SEQ ID NO: 22 - DNA, 25 bp, Artificial
  PCR primer
acaggcacaatgagttgccctatcc

SEQ ID NO: 23 - DNA, 24 bp, Artificial
  PCR primer
atgacagtccaagatgctgcctcc

SEQ ID NO: 24 - DNA, 24 bp, Artificial
  PCR primer
gacctggagtgtgagggaaaagtg

SEQ ID NO: 25 - DNA, 22 bp, Artificial
  PCR primer
tctctgaggaacacggagtatc

SEQ ID NO: 26 - DNA, 24 bp, Artificial
  PCR primer
gctgacagggcaggtttttgtaaa

SEQ ID NO: 27 - DNA, 22 bp, Artificial
  PCR primer
tgtgtcctcagccttgatccat

SEQ ID NO: 28 - DNA, 22 bp, Artificial
  PCR primer
acttattgcagctcctgagcat

(19) United States
(12) Patent Application Publication
(10) Pub. No.: US 2008/0286777 A1
(43) Pub. Date: Nov. 20, 2008

(54) Method of Determining the Diversity of T Lymphocytes in a Biological Sample

(76) Inventors: Serge Candeias, Tullins-Fures (FR); Cedric Touvrey, Grenoble (FR); Evelyne Jouvin-Marche, Meylan (FR); Patrice Marche, Meylan (FR)

Correspondence Address: MORGAN LEWIS & BOCKIUS LLP, 1111 PENNSYLVANIA AVENUE NW, WASHINGTON, DC, 20004, US

(21) Appl. No.: 11/883,633

(22) PCT Filed: Feb. 3, 2006
(86) PCT No.: PCT/FR2006/000247

§ 371 Date: Apr. 1, 2008

(30) Foreign Application Priority Data
Feb. 3, 2005 (FR) 0501073

(51) Int. Cl. C12Q 1/68

(52) U.S. Cl. 435/6

(57) ABSTRACT

The invention relates to the field comprising the diagnosis of possible immune system disorders and the analysis of immune responses. In particular, the invention relates to a method of determining the diversity of T lymphocytes in a biological sample, based on the molecular analysis of the structure of the junctions resulting from the recombination rearrangement V(D)J of element δRec-1 with an AJ gene. More specifically, the invention relates to a method of analyzing the combinatorial diversity and/or the junctional diversity of the excision circles (TREC) resulting from the rearrangement of δRec-1, in order to determine the heterogeneity of a given population of T lymphocytes.

The present invention relates to the field of the diagnosis of possible immune system disorders or disorders which have repercussions on the immune system. The invention relates in particular to a method of determining the diversity of T lymphocytes in a biological sample.

A mature T lymphocyte has at its surface a unique antigen receptor (TCR), formed by the combination of two chains α and β or γ and δ. The TCR performs the function of T lymphocyte antigen recognition, which represents the starting point for the activation and proliferation of these cells. The TCR is expressed clonally: each T lymphocyte carries at its surface a different TCR, specific for a given antigen. The collection of T lymphocytes which have different antigenic specificities, and therefore distinct TCRs, is called a “repertoire”. The analysis of the diversity of TCRs in a given sample therefore makes it possible to determine the diversity of the T lymphocytes in this sample.

The genes encoding the V domain of the TCR chains are formed by the juxtaposition of V and J genes, for the TCRα and TCRγ chains, and V, D and J genes for the TCRβ. and TCRδ chains. They are assembled during T lymphocyte differentiation via a mechanism of directed somatic recombination called “V(D)J recombination”. The TCR genes are grouped together in several loci. The TCRB locus comprises the BV, BD and BJ genes, the recombination of which will give the genes encoding the TCRβ chain. The TCRAD locus is particular: it comprises both the genes encoding the TCRα chain and the genes encoding the TCRδ chain. This locus comprises several tens of ADV/DV genes, a large part of which can be used either for an α chain or for a δ chain. This ADV/DV region is followed by the DD and DJ genes, and then by the AJ genes. A rearrangement between an ADV/DV gene, a DD gene and a DJ gene will encode a TCRδ chain. A rearrangement between an ADV/DV gene and an AJ gene will encode a TCRα chain.

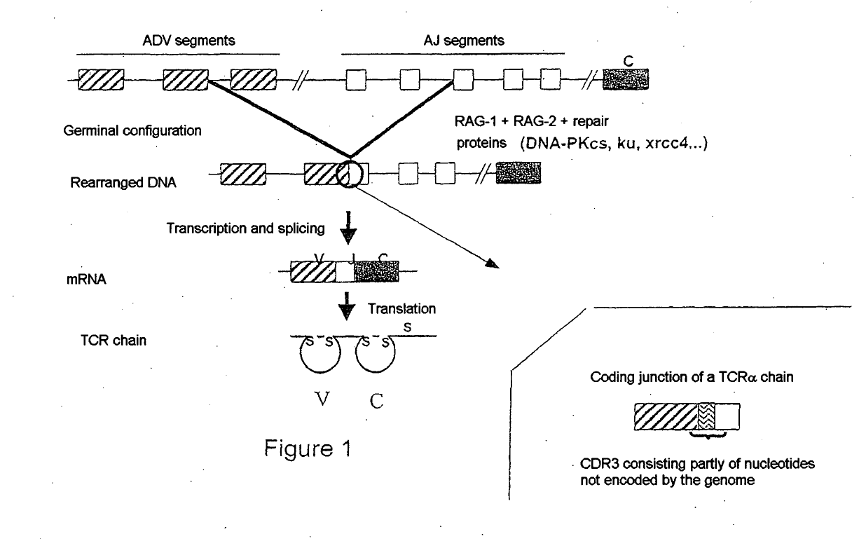

V(D)J recombination makes it possible to generate an extremely vast TCR repertoire. The TCRs differ first of all by virtue of the combination of the rearranged V, D or J segments (combinatorial diversity). For the TCRA genes, encoding the TCRα chain, there are, for example, approximately 100 V genes and 60 J genes in mice. There are therefore potentially 6000 possible combinations (see FIG. 1). For the TCRB genes (encoding the TCRβ chain), there are approximately 450 possible combinations (25 V genes, 2 D genes and 12 J genes).

Furthermore, the molecular mechanisms of V(D)J recombination introduce a junctional diversity. The V, D and J genes are bordered by short conserved nucleotide sequences called recombination signal sequences (or RSSs). These RSSs are nucleotide motifs composed of a conserved heptamer and of a semiconserved nonamer, separated by 12 or 23 bases. The recombination is carried out by a complex which comprises three proteins expressed specifically in lymphocytes: the proteins RAG 1 and 2 (recombination activating genes) and TdT (terminal nucleotidyl transferase). The other enzymatic activities involved in V(D)J recombination are provided by ubiquitous proteins which participate in DNA repair by nonhomologous end joining (NHEJ).

V(D)J recombination is initiated by the introduction of a single-stranded cleavage by RAG proteins bound to the RSSs. This cleavage, located exactly at the junction between the gene and the RSS, generates a free 3′-OH end. A nucleophilic attack on this free end, on the opposite strand, then generates a double-stranded cleavage; the coding end of the gene is closed in a hairpin structure, while the RSS end is blunt and phosphorylated. During the following phase of V(D)J recombination, the coding ends undergo a processing before being joined to one another by the proteins which participate in NHEJ. This involves in particular the opening of the hairpin structure, which is imprecise and can generate inverted repeats of a few bases, called “P nucleotides”. Bases can also be removed from the free ends of the coding DNA. Finally, TdT can add, to these free ends, additional bases called “N nucleotides”, independently of a template.

Consequently, the coding junctions (CJs) exhibit a very great diversity, both from the point of view of the sequence of the junction and of its length. In practice, each junction produced in a T lymphocyte is unique and this junction therefore constitutes the molecular signature of a T lymphocyte. Structurally, within the V domain of TCR chains, the amino acids encoded by the V(D)J junction are at the center of a motif called the “complementary determining region 3” (CDR3), bordered by residues encoded by the V and J genes. Each T lymphocyte expresses a unique combination of TCRα and TCRβ or TCRγ and TCRδ chains, and the CDR3s of these two chains dictate the antigenic specificity of the TCR.

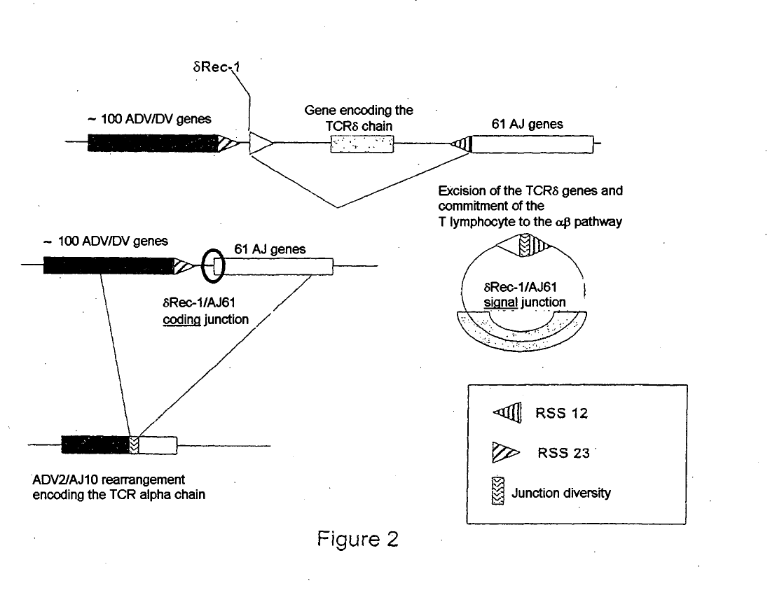

The DNA between the segments fused during the V(D)J recombination is in most cases excised, and its ends comprising the RSSs are joined, thus forming an excision circle (see FIG. 2). These circles, or TRECs (T cell Receptor Excision Circles), do not replicate and are diluted over the course of cell division. Each TREC is therefore the marker of a rearrangement event, and, contrary to the rearrangement encoding the TCR chains, its absolute amount does not vary according to cell proliferation. During the formation of the TRECs, the RSSs are joined by the factors involved in DNA repair by nonhomologous end joining (NHEJ). The junctions resulting from this fusion of the RSSs of the rearranged genes are called “signal junctions” (SJs).

In a few cases, when the rearranged genes are in the reverse transcription orientation, V(D)J recombination does not lead to TREC formation, but results in the inversion of the DNA fragment separating the genes; in this case only, the signal junction is retained on the chromosome and replicated during cell divisions.

When the T lymphocytes differentiate in the thymus, the genes encoding the TCRβ chain are rearranged and expressed before those encoding the TCRα chain. These two periods of rearrangement are separated by an intense proliferation during which the lymphocytes which have succeeded in expressing a TCRβ chain will be greatly amplified (up to 9 division cycles). There can therefore be up to 1000 cells (2⁹) carrying the same TCRB rearrangement, which can potentially rearrange and express different TCRA genes. The ADV and AJ genes are separated on the TCRAD locus by the TCRD genes. In humans, it is generally accepted that during maturation of T αβ lymphocytes, the TCRD genes are excised from the locus before the ADV and AJ genes become rearranged. This excision results from the recombination of a specific RSS, δRec-1, with the closest AJ gene, AJ61. No coding sequence is associated with this RSS. The mechanism of deletion of the gene segments encoding the TCR δ chain, which marks the commitment of the T lymphocytes to the αβ line, has in particular been studied by Shutter et al. (Shutter, Cain et al. 1995), using transgenic mice into which have been integrated the human sequences corresponding to the δ deletion elements and to TCR δ-chain genes. The results described in this study confirm that the δ deletion occurs before the Vα-Jα recombination, and show that this deletion involves the junction of the δRec element with various AJ segments. These authors have also shown, using DNA from a sample of human thymus, that δRec-1 recombines with AJ 61, 60, 59, 58 and 57.

The junctional diversity resulting from the processing of the coding ends is one of the essential characteristics of the V(D)J recombination process, responsible for the vast TCR diversity. On the other hand, it is generally accepted that the RSSs are joined without being processed/modified, and therefore that the signal junctions (SJs) carried by the TRECs are invariant. This idea remains predominantly -widespread, despite some publications describing a diversity at the level of certain SJs. Thus, Candéias et al. have shown that a significant fraction (up to 24%) of the signal junctions resulting from the rearrangement of the TCRB and TCRD genes in mice are modified (Candéias, Muegge et al. 1996). A more recent article, also studying signal junctions resulting from Vβ-Dβ recombinations, shows that the appearance of N nucleotides at the signal junctions apparently depends on the Vβ locus used for the recombination, thereby leading the authors to suggest that the local chromosomal configuration influences the recombinase accessibility (Kanari, Nakagawa et al. 1998). These articles remain, however, limited to the rearrangement of the β chain and, with regard to the article by Candéias et al., also to the rearrangement of the δ chain. Nothing suggests that the mechanisms responsible for the SJ diversity at the level of β and δ chain rearrangements also act with respect to the genes encoding the TCRα chain.

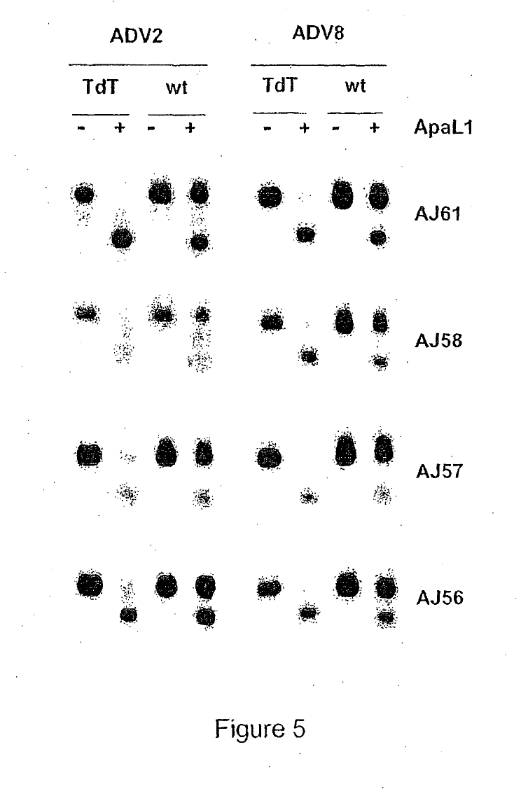

The dominant dogma, according to which the signal junctions do not possess junctional diversity—which dogma is illustrated in particular by FIG. 5 of the recent review article by Jung et al. (Jung. and Alt 2004)—has never been brought into question with regard to the rearrangement of the genes encoding the TCRα chain. On the contrary, several authors have recently described methods of quantifying TRECs resulting from the excision of TCRD genes, in order to evaluate thymic activity in patients, based on real-time amplification of the δRec1/AJ61 signal junction, using a probe covering this signal junction (Hochberg, Chillemi et al. 2001; Schönland, Zimmer et al. 2003), or located just at the border (Hazenberg, Otto et al. 2000). The choice of such a probe indicates that these authors are convinced that this signal junction is invariant. These methods, such as that described by Douek et al. (1998), are implemented in order to quantify the production of new T lymphocytes by the thymus.

In certain situations, it may be advantageous to determine the level of heterogeneity of circulating T lymphocytes. This may be useful for detecting immune system insufficiencies (reduced or no production of new lymphocytes) due to acquired or congenital immune deficiencies. For example, the presence of a clonal or pauciclonal population may be the sign of a developing immune reaction, either against tumor cells, or in response to an infection. It is also useful to determine the level of heterogeneity of circulating T lymphocytes in order to follow the reconstitution of the immune system after a hematopoietic cell graft (bone marrow or stem cells). In fact, this reconstitution leads to the de novo production of lymphocytes, with the possibility of clonal expansion due to graft-versus-host (GvH) or graft rejection reactions. The presence of a clonal or pauciclonal population would then be the sign of the abnormal expansion of a specific lymphocyte pool, thereby possibly revealing a GvH or a rejection.

Several methods aimed at determining the diversity of a T lymphocyte population have already been described. Most of them are reviewed in the review article by Hodges et al. (Hodges, Krishna et al. 2003).

At the cellular level, it is possible to analyze the diversity of a lymphocyte population by flow cytometry (FACS), using a battery of monoclonal antibodies directed against the products of the various Vβ genes (Vβ region). By comparison with normal values, this test makes it possible to determine whether or not the lymphocyte distribution is altered. However, the expression of a Vβ region represents only one of the characteristic parameters of a given lymphocyte since, as set out above, the genes encoding the TCRβ chain are rearranged and expressed before those encoding the TCRα chain, these two periods of rearrangement being separated by an intense proliferation of lymphocytes expressing a TCRβ chain. There exists only a small number of antibodies directed against the products of the Vα genes, making it impossible to analyze this parameter by FACS. The flow cytometry analysis of TRC diversity is therefore, at best, only indicative of the homogeneity of a population of T lymphocytes: it relates only to the expression of the Vβ genes, without even taking into account the junctional diversity of TCRβ chain CDR3s. In addition, this analysis requires a large amount of cells (at least a few million T lymphocytes).

At the molecular level, it is possible to analyze the structure of TCRA and TCRB genes expressed by T lymphocytes by means of various techniques based on PCR, by amplifying the rearranged genes either from transcripts, or from genomic DNA. Since the complete sequence of the TCRB and TCRA loci is available, it is possible to select oligonucleotide primers specific for each BV and ADV gene (or family of genes), for the BJ and AJ genes, and for the BC and AC genes encoding the various domains of the TCRβ and TCRα chains, respectively. Using transcripts, it is therefore possible to analyze the rearrangements of the various ADV and BV families by real-time quantitative PCR. The comparison for each family (41 ADV families and 30 BV families in humans) with the expression profile obtained from control samples makes it possible to determine whether one or more genes are overrepresented, indicating a lymphocyte expansion. This test makes it possible to define the level of expression of each ADV or BV family in a given sample. It does not, however, give any information regarding the diversity of the rearrangements of each ADV or BV gene or gene family.

However, using transcripts, a “immunoscope” test (labeled primer extension) makes it possible to determine the size distribution of the CDR3s of the rearranged TCRA and TCRB genes (international application WO 02/084567). This test requires two PCR reactions to be carried out for each family: the first in order to amplify the transcripts using a given ADV or BV gene and to obtain enough material, the second in order to produce, by extension of a labeled primer, labeled single-stranded DNA which will then be loaded onto an acrylamide gel. This test makes it possible to analyze the heterogeneity of the CDR3s of the TCRα and TCRβ chains expressed in the population analyzed. On the other hand, it does not make it possible to determine the distribution of the various ADV and BV genes in the population analyzed.

The tests described above are relatively laborious to carry out, since they require the preparation of RNA, and then the synthesis of cDNA, from the sample, and also require a large number of PCR reactions to be carried out in order to analyze all the ADV and BV families. In addition, working with RNA requires elaborate precautions to be set up in order to prevent its degradation, and therefore to maintain the representativeness of the sample.

Another method aimed at determining the diversity of circulating T lymphocytes is described in patent application US 2003/0228586. In this application, Sekaly et al. propose to analyze the excision circles generated during the rearrangement of genes encoding the TCRβ chain. The diversity observed by Sekaly et al. is limited to the combinatorial diversity of the rearrangements; the possibility of observing a junctional diversity, during a given rearrangement, is in no way mentioned.

The methods described to date for determining the diversity of a lymphocyte population are therefore all based on the analysis of TCRB and, optionally, TCRA gene rearrangements. None of these methods takes into account the rearrangement which results in the inactivation of the TCRD locus. In this regard, it is known that δRec-1 rearrangement occurs after TCRB gene rearrangement, but before TCRA rearrangement, during T lymphocyte differentiation. The recombination at the level of δRec-1 is considered to be the initiating event for TCRA gene recombination. This rearrangement excises, from the locus, the DD, DJ and DC genes encoding a TCRδ chain and thus prevents the recombination and the expression of the genes encoding a TCRδ chain. It thus definitively commits the T lymphocytes to the αβ pathway. The analysis of this rearrangement would therefore make it possible to specifically determine the diversity of T αβ lymphocytes, while excluding the T γδ lymphocytes.

The inventors have now demonstrated that:
in addition to the rearrangement of δRec1 with an AJ gene, which results in the excision of the entire TCRD locus, other rearrangements exist, resulting in the inactivation of the TCRD genes. The inventors have in particular observed, in mice, δRec-1/DJ2 and DD/AJ rearrangements, which result in the excision, respectively, of all the DD genes and of the DC gene, thus making it impossible for a TCRδ chain to be expressed;
in humans as in mice, δRec-1 rearranges not only with AJ61, but also with (at least) AJ58, AJ57 and AJ56. In humans, δRec-1 also rearranges with at least AJ60 and AJ59;
in humans as in mice, the signal junctions resulting from the rearrangement of δRec-1 with an AJ gene are not invariant. A significant proportion (from 10 to 30%) of the junctions show signs of processing (addition of nucleotides by TdT and/or deletion of a few bases) of the signal ends before joining. As a result of this it is possible to determine a characteristic signal junction heterogeneity profile for each type of δRec-1/AJ rearrangement.

These new results imply that a combinatorial and junctional diversity exists at signal junctions present on TRECs resulting from the excision of TCRD genes. These excision circles therefore constitute a new criterion for analyzing the diversity of T lymphocytes in a sample. This criterion, absent from the current methods for analyzing the diversity of T lymphocytes, described above, is particularly advantageous since TCRD gene excision occurs after TCRB gene rearrangement, but before TCRA rearrangement, during T lymphocyte differentiation. In particular, the inventors have demonstrated that the signal junction produced during δRec-1 recombination constitutes, in the same way as the TCR gene junction, an element of the molecular signature of a T lymphocyte, and that the rearrangement of the δRec-1 element generates a signal junction repertoire with both combinatorial diversity and junctional diversity.

A specific advantage related to the analysis of the signal junctions resulting from the excision of all or part of the TCRD locus is that these junctions are carried by an excision circle which does not replicate during cell proliferation. It does not therefore undergo any of the variations due to the selective elements which shape the repertoire of T lymphocytes according to the antigenic specificity of their TCRs. This rearrangement is therefore a molecular marker characteristic of the emergence of new T lymphocytes in the thymus, before the TCR repertoire is put into place. Such a marker, which is subsequently modified neither qualitatively nor quantitatively, makes it possible to analyze, based on peripheral T lymphocytes, events which have taken place during the emergence of new T lymphocytes in the organism (in particular in the thymus).

The present invention therefore relates, firstly, to a method of determining the diversity of T lymphocytes in a biological sample from a human patient or from an animal, characterized in that it comprises a step of analysis of the combinatorial diversity and/or of the junctional diversity of the excision circles (TRECs) resulting from the excision of all or part of the TCRD locus, during V(D)J recombination. This method can advantageously be carried out by analyzing the combinatorial diversity and/or the junctional diversity of the excision circles (TRECs) resulting from δRec-1 rearrangement.

The patients for whom this method will be particularly useful are those for whom a deficiency of the immune system or a disease with repercussions on the immune system is either suspected or established, and also recipients of a hematopoietic cell transplant, for whom it is desired to follow the reconstitution of the immune system. This method can be used as a complement to the flow cytometry analytical method described above, but it can also be used independently. It makes it possible to analyze, simply, a stage of rearrangement later than TCRB rearrangement, and much less complicated than TCRA rearrangement.

The method of the invention can also be used in animals. An example of implementation in mice is described below, but this method can also be transposed to other animals, in particular to mammals such as bovines, monkeys, pigs, cats, dogs, chickens, etc. The method of the invention may be useful for experimental purposes, for studying animal models of pathologies involving the immune system. In this respect, mention may be made of infections with the simian immunodeficiency virus (SIV) or feline immunodeficiency virus (FIV), which constitute animal models of HIV infection in humans.

Sequence data have been published for certain species of monkeys, and show δRec-1 and AJ RSS sequences very similar to those in humans and in mice. The method of the invention is therefore directly applicable to monkeys. The monkey is used as an animal model for studying various human pathologies. Among these pathologies, mention may be made of infections, in particular with the hepatitis C virus or with the immunodeficiency virus (HIV in humans, SIV in monkeys). In this context, the above method can be used for studying, based on biopsies, the diversity of T lymphocytes present at various stages of the infection, corresponding, for example, to recruitment, then to proliferation of T lymphocytes. The biopsy will be selected by those skilled in the art in such a way as to reflect the evolution of the pathology and/or of the immune response. Thus, for studying the immune response during a hepatitis C virus infection, the biopsies will advantageously be taken from the liver.

The method of the invention can be carried out using any biological sample containing T lymphocytes. By way of nonlimiting examples of biological samples that can be used, mention may be made of whole blood samples, total mononuclear cells, a biopsy containing T lymphocytes, or else a population of T lymphocytes sorted by flow cytometry on the basis of the expression of various membrane markers, including their TCR or a given VR region.

In a preferred embodiment of the method of the invention, the combinatorial diversity of the excision circles resulting from the rearrangement of δRec-1 is analyzed by carrying out at least three amplification reactions on a fragment of the excision circles, each amplification reaction being specific for a signal junction resulting from the rearrangement of δRec-1 with an AJ region that may be joined with δRec-1. The inventors have shown that these AJ regions are essentially the regions AJ61 (also marked ψJα), AJ58, AJ57 and AJ56. There may also be the regions AJ60 and AJ59. Of course, in this embodiment of the invention, the amplification reactions are aimed at rearrangements with at least 3, preferably 4, and possibly 5 or 6 distinct AJ regions. It is important to also note that the analysis of signal junctions corresponding to other rearrangements is not excluded from the methods according to the invention, with a view to a more thorough study of the diversity of a population of T lymphocytes. In fact, δRec-1 can rearrange at least up to AJ18 in mice (signal junctions resulting from this rearrangement have been demonstrated), and at least up to AJ2 in humans (coding junctions resulting from this rearrangement have been demonstrated).

In the above method, the term “amplification reaction” refers to any method of amplifying nucleic acids that is known to those skilled in the art. By way of examples, and in a nonrestrictive manner, mention may be made of PCR (polymerase chain reaction), TMA (transcription-mediated amplification), NASBA (nucleic acid sequence-based amplification), 3SR (self-sustained sequence replication), SDA (strand displacement amplification) and LCR (ligase chain reaction).

According to a preferred embodiment of the method of the invention, the nucleic acid amplification reactions are polymerase chain reactions (PCRs), carried out with pairs of primers each consisting of a primer specific for δRec-1 and a primer specific for an AJ region chosen from AJ61, AJ60, AJ59, AJ58, AJ57 and AJ56. These reactions are preferably carried out in separate tubes. Whatever the technique used to carry out the amplification reactions, the analysis of the results consists in examining which reactions have made it possible to obtain an amplification product. They correspond to the rearrangements present in the population of lymphocytes examined, whereas the absence of amplification of a signal junction between δRec-1 and a given AJ region indicates that the corresponding rearrangement is absent from this population. Of course, positive controls are preferably used when carrying out the method of the invention.

Where appropriate, the amplification can be monitored in real time, for example by carrying out quantitative PCRs. This makes it possible to quantify the TRECs present in the biological sample and, if the latter is appropriate (for example, a blood sample), to evaluate the quantity of T lymphocytes recently produced by the thymus. Methods of quantifying the recent synthesis of T lymphocytes, based on quantifying the TRECs in a biological sample, are in particular described in the articles by Schönland et al., Hazenberg et al., and Hochberg et al., mentioned above. However, in order to quantify the TRECs present in a sample, these authors use a probe located either on the δRec-1/AJ61 signal junction or immediately bordering this junction. Such a probe therefore makes it possible to effectively detect only the nonmodified SJs (in the case of the probe bordering the SJ, it also makes it possible to detect the modified SJs only on the other side of the junction). This may perhaps explain the dispersed nature of the results observed by these authors, even in normal individuals (see in particular FIG. 1 of the article by Hochberg et al.). The use of a probe capable of hybridizing with the TRECs carrying modified SJs will make it possible to reduce this dispersion, and will therefore give results that are easier to interpret by the clinician.

According to one aspect, the present invention therefore proposes a method for evaluating the recent production of T lymphocytes by quantifying the TRECs in an appropriate biological sample, using a probe that is at least 5 to 10, and preferably at least 15, nucleotides away from the signal junction resulting from the perfect rearrangement (without modification) of δRec-1 with at least one AJ region chosen from the group consisting of AJ61, AJ60, AJ59, AJ58, AJ57 and AJ56. This method is advantageously carried out by quantifying the TRECs resulting from the rearrangement of δRec-1 with at least 2, 3 or 4 different AJ regions. This quantification can, of course, be carried out as a complement to the analysis, according to the invention, of the combinatorial and/or junctional diversity of excision circles. According to this aspect of the invention, the distance between the probe and the signal junction is measured relative to the “junction” per se between the two fused RSSs, and therefore relative to the center of the GTGCAC motif resulting from the perfect δRec-1/AJ rearrangement, when this motif is created.

Another important aspect of the invention is the analysis of the junctional diversity at the signal junctions present on the excision circles (TRECs) resulting from the rearrangement of δRec-1. In fact, the inventors have demonstrated that a significant proportion of these signal junctions, ranging up to 40%, exhibit “imperfections”, and in particular additions of N nucleotides. This property, unsuspected before the studies disclosed below, can also be exploited for determining the degree of heterogeneity of a population of T lymphocytes. Thus, the invention also relates to a method for determining the diversity of a population of T lymphocytes present in a sample, comprising at least one step of analysis of the junctional diversity of at least one δRec-1/AJ signal junction. Preferably, the junctional diversity of the most common signal junction, i.e. the δRec-1/AJ61 junction, is examined. More preferably, the analysis of the junction diversity of the SJs is carried out as a complement to the analysis of the combinatorial diversity described above.

As described above, the signal junctions correspond to the fusion of the RSSs bordering the rearranged fragments. These RSSs consist of a conserved heptamer and of a semiconserved nonamer, separated by 12 or 23 bases. The first three bases of the heptamer exhibit a very high degree of conservation, to such an extent that the vast majority of RSSs begin with a CAC triplet. As a result, the “perfect” joining of two RSSs, by NHEJ, during the creation of a signal junction, generates a 5′-GTGCAC-3′ sequence which corresponds to the restriction site for the ApaL1 enzyme. This makes it possible to readily distinguish the signal junctions which have not undergone any addition or deletion, since they are sensitive to restriction with ApaL1, unlike the modified signal junctions.

However, certain RSSs exhibit variations compared with the conserved sequences. This is in particular the case of the RSSs of AJ60 in humans and in mice, and of AJ59 in mice. The analysis of the junctional diversity at the signal junctions involving these RSSs therefore requires the use of other techniques, for example the sequencing of the junctions in question.

According to a preferred embodiment of the methods of the invention involving an analysis of the junctional diversity at the SJs, the analysis of the junctional diversity therefore comprises, for each δRec-1/AJ signal junction analyzed, a step of amplification of a fragment of the excision circle, comprising the signal junction in question. This amplification is preferably followed, for each analyzed δRec-1/AJ signal junction resulting from the rearrangement of δRec-1 with an AJ region chosen from the group consisting of AJ61, AJ59, AJ58, AJ57 and AJ56 in humans, or AJ61, AJ58, AJ57 and AJ56 in mice, by a step of analysis of the ApaL1 restriction profile of the amplified fragment. Various techniques for analyzing a restriction profile of a given DNA fragment, generated by a given enzyme, are well known to those skilled in the art. Of course, the fragment is incubated in the presence of the enzyme in an ad hoc buffer. The result of the digestion can then be analyzed by migration on an agarose gel and visualization using any available technique, for example by staining with ethydium bromide (ETB) or SybrGreen®, or else by blotting onto a membrane and hybridization with a labeled probe (radioactive, coupled to peroxydase, to Texas Red®, or the like).

When the analysis of the junctional diversity takes place as a complement to the analysis of the combinatorial diversity of the TRECs, the same amplification product of a fragment of the TREC can be used for the two aspects.

The interpretation of the result of the digestion of the fragment with ApaL1 is as follows: if the fragment is completely digested, this means that all the SJs corresponding to the δRec-1/AJ junction analyzed are identical and correspond to the nonmodified, “canonic” SJ; if the fragment is completely resistant to the restriction with ApaL1, all the SJs corresponding to the δRec-1/AJ junction analyzed are modified (meaning that they may or may not be identical); the hybrid profile (fragment partially sensitive to ApaL1) is indicative, of the presence, in the sample studied, of at least two populations of lymphocytes comprising TRECs resulting from the rearrangement of δRec-1 with the AJ region analyzed, one comprising a modified SJ, and the other not.

When a modification is demonstrated by the appearance of a fragment resistant to ApaL1, and also for the SJs which do not exhibit an ApaL1 restriction site, even in the absence of modification (for example, the δRec-1/AJ60 SJs), it may be advantageous to carry out a qualitative analysis of the signal junctions, in particular for determining whether or not the population of lymphocytes carrying TRECs resistant to ApaL1 restriction is homogeneous. The term “qualitative analysis” is intended to mean an analysis which makes it possible to determine the nature (addition, deletion, substitution) of the modifications introduced at the ends before they are joined to form the signal junction. Methods as described above, also comprising a step of qualitative analysis of the signal junctions resistant to ApaL1 restriction, are therefore also part of the present invention. This qualitative analysis can be carried out by various techniques known to those skilled in the art, such as sequencing, or labeled primer extension. It is preferably carried out using only the fragments completely or partially resistant to ApaL1, but, where appropriate, with a view to automation, it can be carried out systematically on all the fragments.

A specific method according to the invention comprises the following steps:
a. preparing the DNA from the biological sample;
b. carrying out at least 3 or 4 PCRs, using pairs of primers, each of which consists of a primer specific for δRec-1 and a primer specific for an AJ region chosen from the group consisting of AJ61, AJ58, AJ57 and AJ56;
c. restricting the PCR products with the ApaL1 restriction enzyme;
d. analyzing the restriction profiles obtained in step c;
e. where appropriate, qualitatively analyzing the signal junctions resistant to the ApaL1 restriction, by labeled primer extension.

In step “a” of this method, the preparation of the DNA can be carried out by any technique known to those skilled in the art, such as the techniques described in chapter 6 of the manual by Sambrook and Russel (Molecular Cloning, 3rd edition, CSHL Press), or using commercially available DNA preparation kits..

An intermediate step can, where appropriate, be carried out between steps “b” and “c” of this method, consisting in analyzing the products of the amplifications carried out in step “b”, for example by migration on agarose gel and staining. This makes it possible to limit the analysis of the ApaL1 restriction profiles to only the junctions for which a fragment was effectively amplified.

As mentioned above, the formation of the signal junctions is ensured by ubiquitous proteins which participate in DNA repair. It is. therefore probable that, if one of these proteins is not completely functional, the proportion of modified signal junctions will increase. The methods described above can therefore be used for detecting a possible disorder of the DNA repair mechanism, whether it is a deficiency affecting one of the enzymes involved in repair by nonhomologous end joining (NHEJ), or a factor which acts upstream or downstream of the nonhomologous end joining.

Another aspect of the invention is a kit of reagents for determining the diversity of the T lymphocytes present in a biological sample, by means of a method such as those described above. Such a kit comprises a collection of at least four primers, at least one of which is specific for δRec-1, and at least three of which are each specific for a different AJ region, selected from the group consisting of AJ61, AJ60, AJ59, AJ58, AJ57 and AJ56. According to a specific embodiment, such a kit comprises a collection of at least five primers, at least one of which is specific for δRec-1, at least one of which is specific for AJ61, and at least three of which are each specific for a different AJ region, selected from the group consisting of AJ60, AJ59, AJ58, AJ57 and AJ56. In the kits described above, these primers are of course chosen in such a way as to allow the PCR amplification of the signal junctions resulting from the rearrangement of δRec-1 with the selected AJ regions. Among the additional reagents optionally present in the kits of the invention, mention may be made of the various buffers and/or enzymes required for the amplification reactions and/or for the digestion with ApaL1, samples which can serve as controls for the DNA extraction and/or amplification and/or restriction reactions, etc.

By way of primers and probes that can be used in the methods and kits of the invention, mention may be made of the following oligonucleotides:

TABLE 1

primers and probes for amplifying
signal junctions in humans

| | | |
|---|---|---|
| name | sequence | SEQ ID NO: |

| | | |
|---|---|---|
| hδRec-1RSS | GAAAACACAGTGTGACATGGAGGGCTG | 1 |
| hδRec-1RSSp | AACTCGTGAGAACGGTGAATGAAG | 2 |
| hAJ61RSS | GGTGCCTCTGTCAACAAAGGTGATG | 3 |
| hAJ61RSSp | ATCCCTTTCAACCATGCTGACACC | 4 |
| hAJ60RSS | TCCCCCTTGCATCCCTAATCATGC | 5 |
| hAJ60RSSp | TGAGAGGAGGAAGTTACAGCACAGC | 6 |
| hAJ59RSS | GCAATCAGAGGGCTGAAACACTTGG | 7 |
| hAJ59RSSp | AAAAGGCTGTAAAGATCAAACCACC | 8 |
| hAJ58RSS | TCATATCTCTGGACATATCTGTCAGG | 9 |
| hAJ58RSSp | TAGTTCTTGGGAAGCTCTGCAAAGC | 10 |
| hAJ57RSS | AGTAATCAGATTTGTTCTATAGGTCC | 11 |
| hAJ57RSSp | GGGAATAGCTATACAATTGTATAA | 12 |
| hAJ56RSS | GCTGGTTTTATCAGGGGGATTCTTGG | 13 |
| hAJ56RSSp | TTAAGTAAATATCAAGGGGAGTTTGGG | 14 |

TABLE 2

primers and probes for amplifying
signal junctions in mice

| | | SEQ ID |
|---|---|---|
| name | sequence | NO: |
| 5′JATA47 | CAGTAGGGGATGGATGCTAACATGA | 15 |
| (AJ56) | | |
| 5′AJ47p | AGTCCACAGATCCTACCACTGCTG | 16 |
| (AJ56) | | |
| AJ57RSS-L | TCCCTGGGAGACTCAC | 17 |
| AJ57RSSp | TCTGGCTCCTATCGGCTAGCAGAAG | 19 |
| AJ58RSS-L | GGATGGTATCGCTTATTCCT | 19 |
| AJ58RSSp | TCGCACAGTGGAGGAAACTTCTAGTCCT | 20 |
| AJ59RSS | CTCAGCAGACCTCAGTCCATCACC | 21 |
| AJ59RSSp | ACAGGCACAATGAGTTGCCCTATCC | 22 |
| AJ60RSS | ATGACAGTCCAAGATGCTGCCTCC | 23 |
| AJ60RSSp | GACCTGGAGTGTGAGGGAAAAGTG | 24 |
| TREC AJ61 | TCTCTGAGGAACACGGAGTATC | 25 |
| TREC AJ61p | GCTGACAGGGCAGGTTTTTGTAAA | 26 |
| TREC DR | TGTGTCCTCAGCCTTGATCCAT | 27 |
| TREC DRp | ACTTATTGCAGCTCCTGAGCAT | 28 |

In Tables 1 and 2 above, the oligonucleotides whose name ends with a “p” can be used as probes when they are labeled, but can also be used as primers, in particular for carrying out nested or seminested PCRs (i.e. from a product amplified by PCR, using two or one primer(s), respectively, internal to the product of the first amplification).

In addition to the above provisions, the invention also comprises other provisions which will emerge from the description which follows, which refers to examples of implementation of the method which is the subject of the present invention and also to the attached drawings, in which:

FIGS. 1 and 2: explanatory schemes of V(D)J recombination.

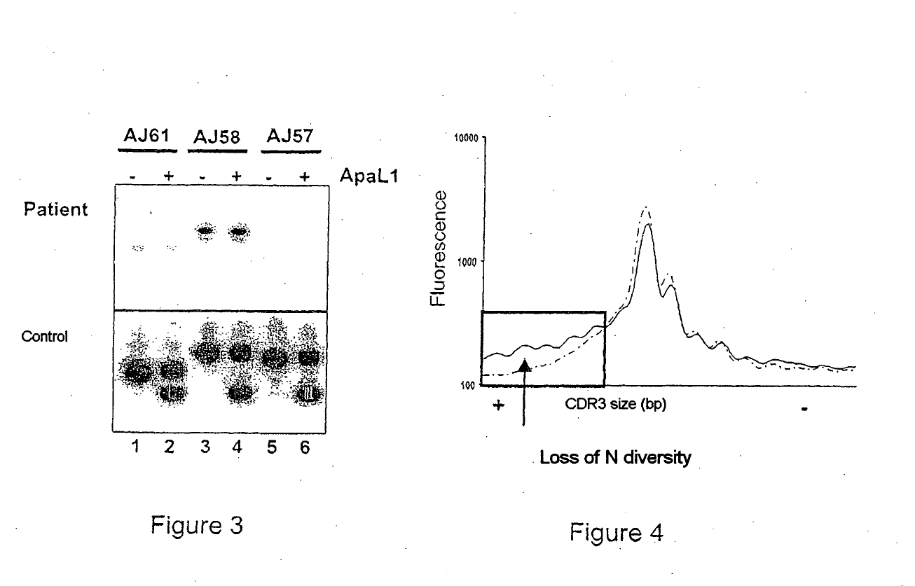

FIG. 3: analysis by amplification and ApaL1 digestion of the signal junctions present in a population of T lymphocytes isolated from a patient.

The δRec-1/AJ61, δRec-1/AJ58 and δRec-1/AJ57 signal junctions were amplified from DNA extracted from the CD4+CD8+Vβ1+ peripheral lymphocytes (sorted by FACS) of a patient and a sample of human thymus (polyclonal control). The undigested (lanes 1, 3 and 5) and ApaL1-digested (lanes 2, 4 and 6) PCR products were separated on an agarose gel, and then blotted onto a nylon membrane and hybridized with a radioactive probe specific for δRec-1.

For the patient, only the δRec-1/AJ61 and δRec-1/AJ58 amplifications (lanes 1 and 3) give a product, whereas, for the control, the 3 reactions are positive (lanes 1, 3 and 5). The diversity of the δRec-1 signal junction repertoire is therefore reduced in the patient. Furthermore, the amplified δRec-1/AJ61 and δRec-1/AJ58 signal junctions from the patient are completely resistant to the digestion with the ApaL1 restriction enzyme (lanes 2 and 4), thereby indicating that the signal ends of the rearranged genes were reorganized before being joined. On the other hand, all the signal junctions amplified from the control DNA contain both products sensitive and products resistant to the digestion (lanes 2, 4 and 6). The amplified junctions from the patient are therefore less diversified than in the control sample, since they do not comprise any nonmodified junctions. The CD4+CD8+Vβ1+ lymphocytes isolated from the patient thereby have a reduced δRec-1 rearrangement repertoire: there are fewer δRec-1 rearrangements, and their junctional diversity is restricted, compared with a control polyclonal sample.

FIG. 4: analysis of the δRec-1/AJ58 junction diversity

In order to determine whether the δRec-1/AJ58 PCR product (resistant to digestion with ApaL1) amplified from the CD4+CD+Vβ1+ population of the patient contains one or more molecular species, an analysis of diversity was carried out. The δRec-1/AJ58 PCR product was used as a template in a PCR reaction intended to produce labeled single-stranded DNA using a δRec-1 primer coupled to Texas Red®. This single-stranded DNA was then loaded onto an acrylamide gel, with the corresponding product amplified from the control sample. This method makes it possible to separate the various molecular species according to their lengths, each size giving a fluorescence peak. In the control sample (solid-line curve), a predominant peak is observed at the size expected for a nonmodified junction. These molecular species correspond in large part to the junctions sensitive to digestion with ApaL1. Several peaks corresponding to larger sizes, which represent molecular species comprising junctions modified at least by addition of nucleotides, are also observed (boxed in). These peaks correspond to the modified junctions, present in this PCR product, which are resistant to the digestion with ApaL1.

In the δRec-1/AJ58 PCR product amplified from the CD4+CD8+Vβ1+ population of the patient, a single peak is observed. This product therefore contains only a single molecular species. It migrates to the position corresponding to the size expected for a nonmodified junction, although it is resistant to the digestion with ApaL1 (see FIG. 1). It can therefore be concluded from this that this. junction has been modified by deletion and then addition of nucleotide(s).

FIG. 5: demonstration of N modifications in ADV/AJ sJs

The signal junctions resulting from rearrangements of ADV2 and ADV8 with AJ61, AJ58, AJ57 and AJ56 were amplified from thymocyte DNA from C57BL/6 mice (wt) and TdT-deficient mice (TdT). The PCR products were undigested (−) or digested (+) with ApaL1 before migration on a gel and hybridization with internal AJ, probes.

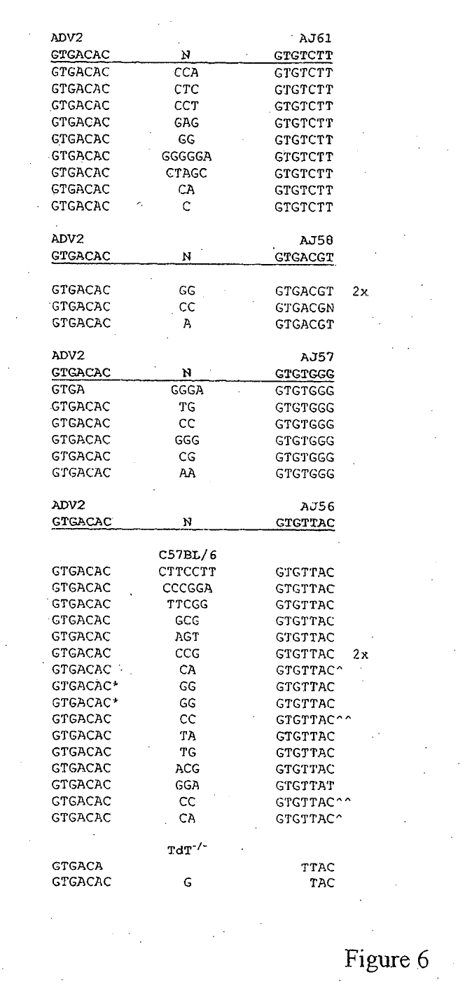

FIG. 6: result of the sequencing of the ADV2/AJ signal junctions from samples of wild-type mice and TdT^{0/0} mice.

* indicates two sequences with the same junction but using different ADV2 genes; ̂ and ̂̂ indicate two sequences with the same junction, amplified from DNA of thymocytes from two different mice.

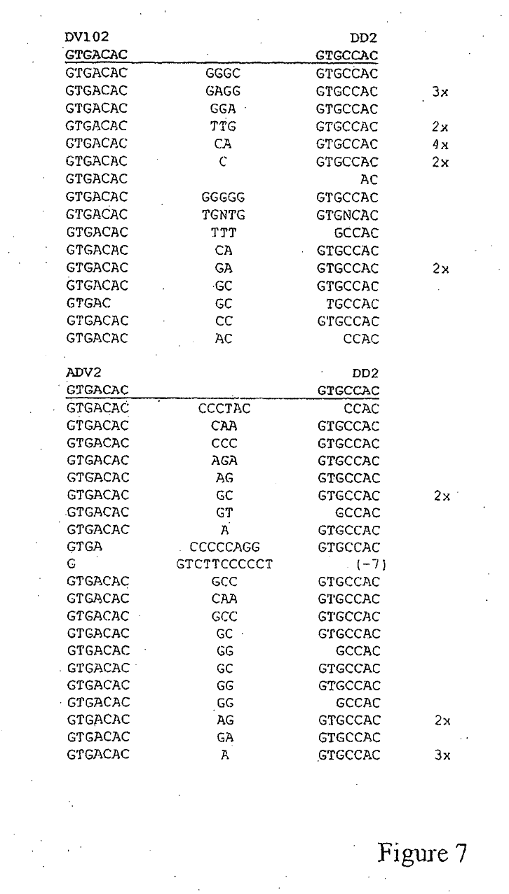

FIG. 7: result of the sequencing of DV102/DD2 and ADV2/DD2 murine signal junctions.

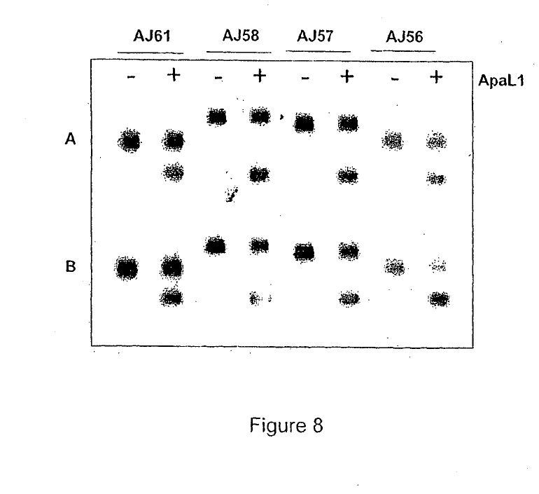

FIG. 8: analysis of human δRec-1/AJ signal junctions.

Samples of DNA from thymocytes of two patients aged 10 days (A) and 6 days (B) were used to amplify the SJs resulting from the rearrangement of δRec-1 with AJ61, AJ58, AJ57 and AJ56. A fraction of the PCR products was digested with ApaL1; the digested and undigested products were then separated on an agarose gel, blotted onto a nylon membrane, and hybridized with a radiolabeled δRec-1 probe.

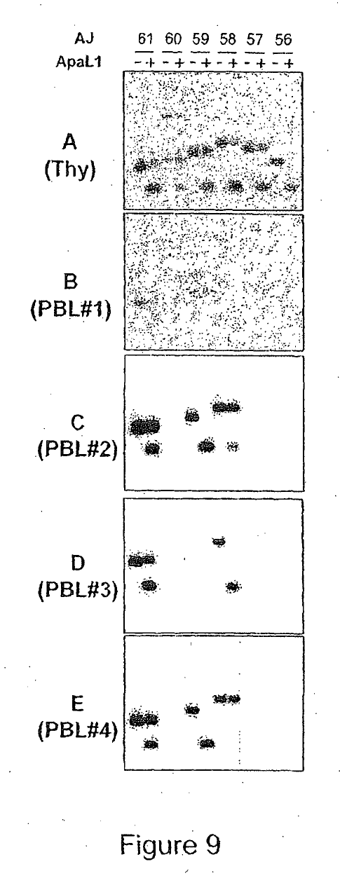

FIG. 9: amplification and analysis of the signal junctions produced during the recombination of hδRec-1 with the AJ61, 60, 59, 58, 57 and 56 genes in a sample of thymocyte DNA (A) and 4 samples of DNA from leukocytes purified from the blood (B, C, D and E) of 4 different patients.

The signal junctions were amplified by PCR and then analyzed on an agarose gel without (−) or after (+) prior digestion with the ApaL1 restriction enzyme. After blotting onto a nylon membrane, the products are revealed by hybridization of a radioactive probe specific from hδRec-1.

For the Thy and PBL#1 samples (A and B), the asterisk indicates the position of a band corresponding to an hδRec-1/AJ59 signal junction, which can sometimes be amplified with the oligonucleotide specific for AJ60 due to the proximity of these genes.

The profile of the products obtained is different in each sample of blood leukocytes, indicating the diversity of the repertoires in these patients.

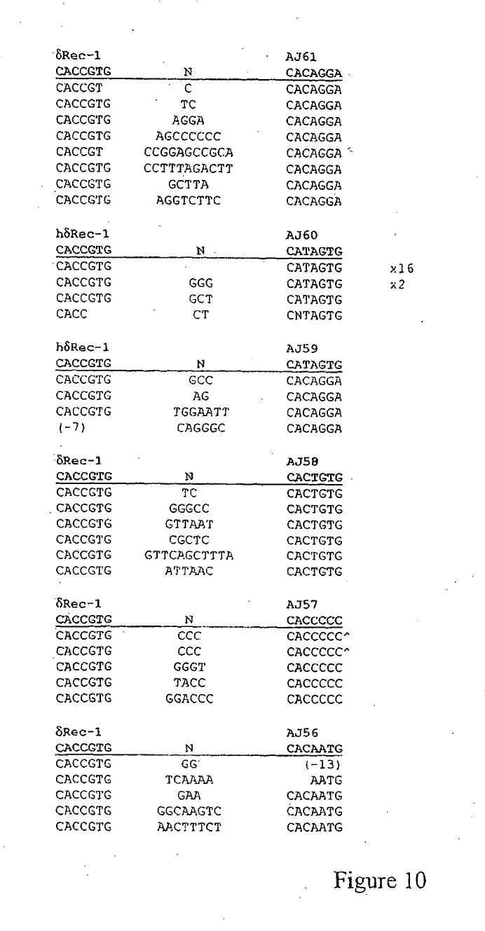

FIG. 10: result of the sequencing of the δRec-1/AJ signal junctions resistant to ApaL1 restriction, for the rearrangements of hδRec-1 with AJ61, AJ59, AJ58, AJ57 and AJ56, and of the total signal junctions, for the hδRec-1/AJ60 rearrangements present in a human sample.

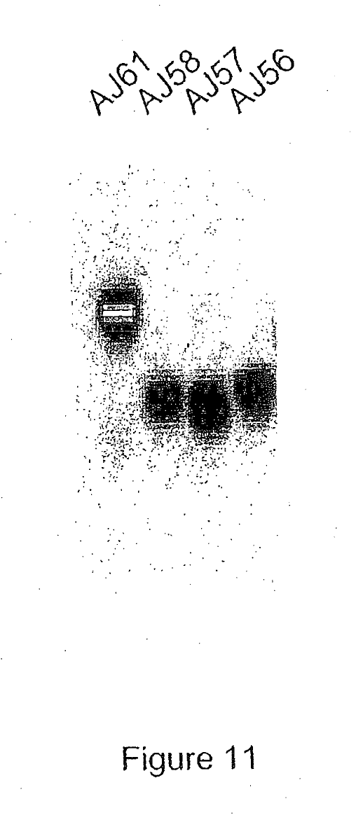

FIG. 11: amplification of the SJs resulting from the rearrangement of δRec-1 with AJ61, AJ58, AJ57 and AJ56, using murine thymocyte DNA.

The PCR products were separated on an agarose gel and then blotted onto a nylon membrane and hybridized with a radiolabeled δRec-1 probe.

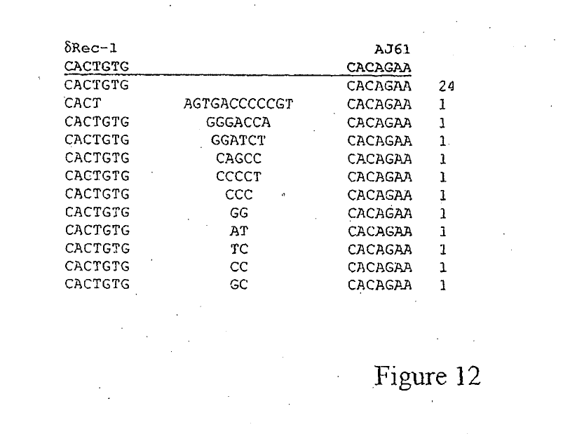

FIG. 12: result of the sequencing of the δRec-1/AJ61 signal junctions present in a murine sample.

It should be clearly understood, however, that these examples are given only by way of illustration of the subject of the invention, of which they in no way constitute a limitation.

### EXAMPLES

### Example 1

### Determination of the Degree of Heterogeneity of a Population of T Lymphocytes from a Patient

### 1.A. Procedure

1.A.1. Preparation of DNA

For the DNA extraction, the nucleospin tissue extraction kit from Machery Nagel® was used, according to the instructions provided.

The cells are pelleted by centrifugation for 5 minutes at 1200 rpm (corresponding to 300 g). The dry pellet (up to 10 million cells) is taken up in 185 μl of T1 lysis buffer mixed with 25 μl of proteinase K provided in the kit.

The sample is vigorously vortexed and digested overnight at 56° C.

200 μl of buffer B3 are then added, and the lysate is vortexed and incubated at 70° C. for 10 minutes.

210 μl of absolute ethanol are added and the mixture is loaded onto a column provided in the kit.

The column is vortexed at 11,000 g for one minute (binding of the DNA to the silica).

The column is then washed with 500 μl of BW and then 600 μl of B5 with centrifugation for 1 minute at 11000 g each time.

The silica is dried by centrifugation for 3 minutes at 11000 g.

The DNA is then eluted with a volume of 50 to 200 μl of BE according to the amount of cells lysed at the beginning. For this, the BE preheated to 70° C. is loaded onto the column and the whole is incubated for 2 minutes at 70° C. in an incubator. DNA is recovered by centrifugation for 1 minute at 11,000 g.

1.A.2. Signal Junction Amplification

The aim of this step is to amplify, by PCR, the signal junctions of interest in order to obtain enough material to study their structure.

100 to 200 ng of total DNA are used for each PCR reaction. Depending on the sample, a second PCR (nested PCR) may be necessary.

The amplifications are carried out using the TaqGold enzyme (Applied Biosystem), in the “Master Mix” buffer provided. Primers specific, firstly, for δRec-1 (SEQ ID No: 1) and, secondly, for AJ61 (SEQ ID No: 3), AJ 58 (SEQ ID No: 9), AJ57 (SEQ ID No: 11) and AJ56 (SEQ ID NO: 13) were used. These primers are chosen so as to amplify the signal junctions only. Four PCR reactions are carried out per sample.

For 25 μl of reaction:

| | |
|---|---|
| DNA: | 100 or 200 ng |
| 2 × Master Mix: | 12.5 μl |
| sense oligo at 5 μM: | 2 μl |
| reverse oligo at 5 μM: | 2 μl |
| H₂O: | qs 25 μl |
| PCR conditions: | 10′ at 94° C. |
| | 35 cycles comprising the following |
| | sequence: |
| | 30″ at 94° C., 30″ at 64° C. and 30″ at |
| | 72° C., |
| | then |
| | 10′ at 72° C. |

Second PCR with a more internal oligonucleotide specific for the AJ region (nested PCR): SEQ ID No: 4 for AJ61, SEQ ID No: 10 for AJ58, SEQ ID No: 12 for AJ57 and SEQ ID No: 14 for AJ56, and, still, SEQ ID No: 1 for δRec-1.

For 25 μl of reaction:

| | |
|---|---|
| product from the 1st PCR: | 4 μl |
| 2 × Master Mix: | 12.5 μl |
| sense oligo at 5 μM: | 2 μl |
| reverse oligo at 5 μM: | 2 μl |
| H₂O: | qs 25 μl |
| PCR conditions: | 10′ at 94° C. 1X |
| | 35 cycles comprising the following |
| | sequence: |
| | 30″ at 94° C., 30″ at 64° C. and 30″ at |
| | 72° C., |
| | then |
| | 10′ at 72° C. |

The PCR products are loaded onto an agarose gel. A first indication of the degree of heterogeneity of the lymphocytes contained in the sample is obtained at this stage, according to whether or not the 4 reactions are positive.

1.A.3. Detection of the Diversity of the Signal Junctions by Digestion with the ApaL1 Enzyme:

The aim of this step is to determine whether the signal junctions amplified in the previous step are diversified. The RSSs of the selected genes (δRec-1, AJ61, AJ58, AJ57, AJ56) correspond to the consensus RSS sequence and therefore begin with the 3 nucleotides GTG or CAC. Consequently, the perfect joining of the two RSSs so as to form a signal junction creates a site (5′-GTGCAC-3′) recognized by the ApaL1 restriction enzyme).

A large fraction (up to 30%) of the signal junctions resulting from the V(D)J recombination of the genes carried by the TCRAD locus are not formed by perfect joining of the RSSs of the rearranged genes, but show signs of processing by deletion and/or addition of nucleotides at the RSS ends before joining. The junctional diversity which results therefrom prevents the formation of the ApaL1 restriction site, and these modified junctions are therefore resistant to digestion with this enzyme. The presence of two molecular species after digestion of the PCR products with the ApaL1, one sensitive and one resistant, is therefore indicative of the diversity of the amplified junctions.. This diversity reveals the presence of T lymphocytes having performed different V(D)J recombination events.

If, on the other hand, only one molecular species is. detected, then the diversity of the sample analyzed is reduced compared with a control sample.

The digestion is carried out in a final volume with 20 μl at 37° C. for 3 h with 5 U of ApaL1 enzyme.

| | |
|---|---|
| PCR product: | 10 μl |
| 10 × digestion buffer: | 2 μl |
| 10 U/μl enzyme: | 0.5 μl |
| H₂O: | 7.5 μl |

A control is carried out under the same conditions, omitting the restriction enzyme:

| | |
|---|---|
| PCR product: | 10 μl |
| 10 × digestion buffer: | 2 μl |
| H₂O: | 8 μl |

The digested or undigested products are separated by electrophoresis on a 2% agarose gel in 1X TBE buffer in the presence of ETB.

The DNA is then transferred by capillary action onto a nylon Hybond N+ membrane (Amersham), and then fixed under UV (700 J).

The membrane is then prehybridized with Rapid Hyb buffer (Amersham) for 30 min at 42° C., and then hybridized at 42° C. for 4 hours with an oligonucleotide probe specific for δRec-1, labeled with ³²P by incubation with the T4 phage polynucleotide kinase and ATPγ³²P, under the conditions indicated by the supplier.

The radioactive signal is detected using a phosphor imager.

1.A.4. Qualitative Analysis of Human Signal Junctions by Labeled Primer Extension

This technique makes it possible to determine the length of the various δRec-1/AJ signal junctions amplified from a population of T lymphocytes. It is thus possible to qualitatively determine the modifications introduced into the signal junction. The distribution of the fragments obtained makes it possible to estimate the diversity of the population analyzed. This method consists in amplifying single-stranded DNA by PCR with a primer containing a fluorochrome: Texas-red. These fragments are then resolved on a polyacrylamide gel.

| | |
|---|---|
| Final volume: | 12.5 μl with the amplitaq Gold master Mix |
| | kit from Applied Biosystems |
| PCR product: | 0.5 μl |
| 2 × Master Mix: | 6.25 μl |
| 1 μM oligo: | 0.5 μl |
| H₂O: | 5.25 μl |
| PCR cycles: | 6′ at 94° C. |
| | 12 cycles comprising the following |
| | sequence: |
| | 2′ at 94° C., 1′ at 64° C. and 7′ at |
| | 72° C., then |
| | 12′ at 72° C. |

9 μl of PCR product were then mixed with 16 μl of formamide and 1 μl of fluorescent label. The mixture was heated at 96° C. for 5′ and then injected into an ABI Prism 310 sequencer.

### 1.B. Results

The flow cytometry analysis of the T lymphocytes in blood samples from a patient showed a very altered distribution of the use of the various Vβ genes. The T lymphocytes from this patient use very predominantly the Vβ1 gene. Furthermore, an unusual population of T lymphocytes, which expresses both CD4 and CD8 molecules, was detected in this patient. The T lymphocytes of this population also use virtually exclusively the Vβ1 gene.

These CD4+CD8+Vβ1+ T lymphocytes were sorted by flow cytometry and their DNA was prepared. This DNA was used as a template to amplify the δRec-1/AJ61, δRec-1/AJ58 and δRec-1/AJ57 signal junctions (the fourth combination, δRec-1/AJ56, was not carried out in this example). Only the δRec-1/AJ61 and δRec-1/AJ58 amplifications gave a product, whereas a control sample of DNA extracted from total thymocytes (normally diversified population) gave a product for the 3 reactions (FIG. 1, lanes 1, 3 and 5). This result indicates that the CD4+CD8+Vβ1+ T lymphocytes purified from the blood of the patient have a restricted δRec-1 rearrangement repertoire.

The PCR products were subjected to digestion with the ApaL1 restriction enzyme and those originating from the patient were found to be resistant (FIG. 1, lanes 2, 4 and 6). This result indicates that all the amplified signal junctions are modified in the patient, whereas in the control sample amplified from thymocytes, the majority of the signal junctions are nonmodified, and products sensitive to and products resistant to digestion with ApaL1 are therefore distinguished (FIG. 1, lanes 2, 4 and 6). This result indicates that the junction diversity of the signal junctions amplified from the patient's CD4+CD8+Vβ1+ T lymphocytes is limited.

In order to determine whether the signal junctions amplified from the CD4+CD8+Vβ1+ population are polyclonal or monoclonal, a labeled primer extension test of immunoscope type was carried out on the δRec-1/AJ58 PCR product. A single molecular species was found in this PCR product (FIG. 2).

These experiments indicate 1) that the repertoire of δRec-1 rearrangements is limited in the population analyzed (only 2 rearrangements out of 3 tested), and 2) that the junctions which are found exhibit a very restricted degree of diversity compared with a control polyclonal sample.

These results therefore suggest that the population of CD4+CD8+Vβ1+ T lymphocytes results from the proliferation of a small number of initial lymphocytes.

### Example 2

### Modification of the Signal Junctions during V(D)J Recombination of the Genes of the TCRAD Locus in Mice

### 2.A. Materials and Methods

2.A.1. Mice

C57BL/6 mice were reared in the animal house of the Commissariat à l'Energie Atomique [Atomic Energy Commission] in Grenoble. The mice were sacrificed by inhalation of CO₂ and their thymus was removed at the age of 4-8 weeks.

2.A.2. Preparation of DNA

The DNA of C57BL/6 mouse thymocytes was prepared from 10⁷ cells using the Machery Nagel® nucleospin tissue extraction kit, by following the manufacturer's instructions. The DNA of TdT^{0/0} thymocytes was obtained from TdT^{0/0} mice (Gilfillan, Dierich et al. 1993).

2.A.3. Amplification and Digestion of Signal Junctions

The thymocyte DNA (100 ng) was amplified with appropriate primers using the AmpliTaqGold PCR mixture as follows: 10 minutes at 94° C., then 35 cycles comprising the following sequence: 30 sec at 94° C., 30 sec at 60° C. and 30 sec at 72° C., followed by 10 min at 72° C. The PCR reactions were carried out on a Perkin-Elmer “GenAmp PCR system 9600” machine, in a final volume of 25 μl. The primer sequences are indicated in tables 1 and 2 above.

After amplification, 5 to 10 μl of the PCR amplification products were digested with 5 units of ApaL1 restriction enzyme (Amersham Pharmacia Biotech) for 3 hours at 37° C. in the buffer supplied with the enzyme. Controls were incubated in the same way, but without ApaL1. The digested and undigested products were resolved side by side on a 2% agarose gel, blotted onto an N-Hybond+ membrane (Amersham Biosciences) and hybridized with a mixture of radiolabeled oligonucleotide probes specific for the AJ regions of the amplified signal junctions presented in tables 1 and 2 above (probes of SEQ ID Nos: 16, 18, 20 and 24). The analysis of the blots hybridized with these probes was carried out on a “Personal FX Imager” (Biorad), using the “Quantity One” software.

2.A.4. Cloning and Sequencing of Signal Junctions

The PCR products were purified on a gel, cloned into the vector pGEM-T Easy (Promega®) according to the manufacturer's instructions, and transformed into competent bacteria. After plating out, the positive colonies were identified by hybridization with oligonucleotide probes specific for the AJ regions. The plasmids were prepared, from the colonies containing the signal junctions, either with the Wizard miniprep kit (Promega®) or with the Montage Plasmid Miniprep 96 system (Millipore Corporation®). The plasmids containing the SJs were then individually digested with ApaL1. The plasmid pGEM-T easy contains 2 sites for this enzyme. If the recombinant plasmid contains a nonmodified signal junction, formed by perfect fusion of the RSSs, an additional ApaL1 site is introduced and the digestion gives 3 bands after migration on an agarose gel. If the recombinant plasmid contains a modified signal junction, such an additional site is not introduced and the digestion gives only 2 bands.

The plasmids showing unexpected profiles, in terms of the number or the size of the DNA fragments, were excluded from the subsequent analyses. The plasmids containing modified signal junctions were sequenced (Genome Express, Meylan, France), in order to identify the nature of the modifications. The identical signal junctions obtained from the same sample were counted only once, since it is impossible to determine whether these multiple occurrences are the result of independent events or of an overamplification of a single signal junction.

### 2.B. Results

2.B.1. The ADV/AJ Signal Junctions Exhibit a Junctional Diversity Linked to the Action of TdT

The SJs resulting from the recombination of the ADV2 and ADV8 genes with AJ61, AJ58, AJ57 and AJ56 were amplified from C57BL/6 mouse thymocyte DNA. The undigested and ApaL1-digested PCR products were subsequently analyzed by migration on an agarose gel, followed by revelation by blotting and hybridization with radiolabeled probes specific for the AJ regions. For all the ADV-AJ combinations tested, a significant fraction of the PCR products was resistant to ApaL1 restriction. This resistance indicates that some signal junctions are modified and do not consist of a simple joining of the RSSs.

The potential involvement of TdT in the generation of the signal junctions resistant to ApaL1 restriction (SJ ApaL1-R) was analyzed by carrying out the same, experiments using DNA from TdT-deficient thymocytes. This revealed clearly that, in the absence of TdT expression, the SJ ApaL1-R are virtually nonexistent, for all the combinations tested (FIG. 5).

Similar results were obtained by analyzing the SJS produced after the rearrangement of ADV1, ADV20 and ADV6 with the same AJ genes. The SJ modification therefore occurs during the rearrangement of at least 5 of the 21 ADV gene families located in the ADV/DV region 5′ of δRec-1.

These results show that (i) the ADV-AJ signal junctions exhibit a junctional diversity, and (ii) this diversity results almost completely from the addition of N nucleotides by TdT.

2.B.2. Quantification and Structure of the Modified Signal Junctions

In order to accurately determine the percentage of modified signal junctions, the ADV2/AJ signal junctions were amplified from wild-type thymocyte DNA and cloned, in such a way that each SJ can be analyzed independently by ApaL1 digestion of the corresponding recombinant plasma. As shown in table 3 below, the frequency of the SJ ApaL1-R ranges from 9.7% for ADV2/AJ58 to 39.1% for ADV2/AJ61, with an average of 33.6%. Thus, a significant fraction of the ADV2/AJ SJs are modified, even if the frequency of the modifications appears to vary slightly according to the AJ gene rearranged.

TABLE 3
analysis of the signal junctions produced
during the rearrangement of the TCRA genes in mice

| | | | | Unique | Frequency | | SJ |
|---|---|---|---|---|---|---|---|
| | ApaL1-S | | ApaL1-R | sequences | R*/(R* + | | with |
| | (S) | ApaL1-R | sequenced | (R*) | S) | N/Seq | deletion |
| ADV2- | 14 | 11 | 9 | 9 | 39.1% | 3.1 | 1 |
| AJ61 | | | | | | | |
| ADV2- | 28 | 4 | 4 | 3 | 9.7% | 1.7 | 0 |
| AJ58 | | | | | | | |
| ADV2- | 25 | 6 | 6 | 6 | 19.3% | 2.5 | 0 |
| AJ57 | | | | | | | |
| ADV2- | 35 | 18 | 17 | 16 | 31.4% | 3.1 | 0 |
| AJ56 | | | | | | | |
| Total | 67 | 39 | 36 | 34 | 33.6 | 2.6 | 1 |

The majority of these SJ ApaL1-R were sequenced in order to precisely identify the nature of the modifications. These modifications consisted essentially of the addition of N nucleotides. It was found that these sequences contained between 1 and 7 nucleotides without support on a template, with an average of 2.8 per sequence. These additions of N nucleotides correspond mainly to the nucleotides of G and C, which together represent more than 70% of the bases added (FIG. 6). This constitutes a typical bias of the TdT activity. Apart from two exceptions, the ApaL1-resistant ADV2/AJ SJs were unique, thereby indicating that the repertoire of the modified SJs exhibits great diversity.

It is important to note that an ADV2/AJ57 SJ into which 4 N nucleotides have been inserted also shows a deletion of 3 bases of the ADV2 heptamer, thereby suggesting that a loss of nucleotides from the RSS ends can contribute to the SJ diversity. This observation led the inventors to examine the structure of the SJs in the absence of TdT expression. ADV2/AJ56 SJs amplified from TdT-deficient mice were cloned, and the recombinant plasmids obtained were analyzed. The frequency of the ApaL1-R plasmids was found to be 2.7% (2 out of 74). In one of these sequences, nucleotides had been deleted from the two signals carried on ADV2 and AJ56 (1 and 3 nucleotides, respectively). In the other, 4 bases had been deleted from the AJ56 heptamer, whereas the signal on ADV2 is complete. This junction also contains an additional nucleotide (FIG. 6, last lines).

It therefore appears that the loss of bases from the RSSs is rare, and that most of the modifications observed in the SJs of TCRA result from additions of N nucleotides.

2.B.3. The Signal Junctions of TCRD are Modified by Additions of N Nucleotides and by an Exonucleolytic Activity

A previous study by Candéias et al. (above) showed that TCRD gene rearrangement generates signal junctions modified both by addition of N nucleotides and by loss of base(s). These data were obtained after analysis of the rearrangement of DV105 with DD2. However, DV105 rearranges by inversion and SJ formation is therefore essential for maintaining chromosomal integrity and ensuring cell survival. It is theoretically possible that, during recombination by inversion, the RSSs are more tightly linked with the V(D)J recombination complex, including the exonucleolytic activity, in order to ensure formation of the SJ. This could explain the presence of deletions at the RSS ends of DV105 and DD2. Consequently, in order to determine whether the deletion of bases of the signal elements (SEs) is a general characteristic of TCRD gene rearrangement, or whether, on the contrary, this constitutes a specificity of rearrangement by inversion, the inventors analyzed the structure of the SJs produced during the rearrangement which occurs by deletion between DV102 and DD2.

ApaL1 digestion of the recombinant plasmids obtained after amplification of the DV102/DD2 SJs and cloning from C57BL/6 mouse thymocyte DNA revealed the presence of 34% of SJ ApaL1-R (table 4). Notably, the sequencing analysis showed that four of these modified signal junctions (out of 17 different junctions) lost nucleotides at the terminal ends of the RSSs of DV102 and/or DD2 (FIG. 7). The loss of nucleotides from the SEs before joining thereof is not therefore dependent on whether the TCRD genes are rearranged by deletion or inversion.

The same analysis was then carried out on the ADV2/DD2 SJs amplified from C57BL/6 thymocyte DNA. The proportion of ApaL1-R recombinant plasmids was found to be 44.7% (table 4). The sequencing analysis revealed 21 different modified junctions. In 5 of them, the modifications consisted both of the addition of N nucleotides and of the deletion of bases from the signal ends (FIG. 7). This proportion is similar to that found in the DV102/DD2 SJs. However, it is statistically different (p=0.01 by the Mann-Whitney test) than that of the deletions found in the ApaL1-resistant ADV2/AJ SJs (1 deletion out of 34 signal junctions in wild-type thymocytes), thereby indicating that further deletions are introduced into the SJs produced during TCRD gene rearrangement than during TCRA gene rearrangement, even when the ADV gene family is used in the two types of rearrangements.

TABLE 4
analysis of the signal junctions produced
during TCRD gene rearrangement in mice

| | | | | Unique | Frequency | | SJ with |
|---|---|---|---|---|---|---|---|
| | ApaL1-S | | ApaL1-R | sequences | R*/(R* + | | deletion |
| | (S) | ApaL1-R | sequenced | (R*) | S) | N/Seq | (s) |
| ADV2- | 26 | 28 | 25 | 21 | 44.7% | 2.8 | 6 |
| DD2 | | | | | | | |
| DV102- | 33 | 25 | 25 | 17 | 34% | 3.1 | 4 |
| DD2 | | | | | | | |
| Total | 59 | 53 | 50 | 38 | 39.2 | 2.9 | 10 |

It therefore appears that the mechanisms responsible for signal junction diversity differ according to whether the SJs in question result from the rearrangement of TCRA genes or TCRD genes.

2.B.4. Rearrangement of the δRec-1 Element Exhibits Combinatorial and Junctional Diversity

The inventors subsequently analyzed in greater detail the structure of the signal junctions produced by the recombination of the δRec-1 element with the AJ genes. The SJs produced by the recombination of δRec-1 with AJ61, AJ60, AJ59, AJ58, AJ57 and AJ56 were amplified from human thymocyte DNA, and the products of these amplifications were digested with ApaL1. FIGS. 8 and 9A show that, in the thymus, a considerable fraction of the PCR products is resistant to ApaL1 restriction, thereby revealing the existence, for all the rearrangements tested except those involving AJ60, modified signal junctions. As regards the hδRec-1/AJ60 signal junctions, all these junctions are resistant to digestion with the ApaL1 restriction enzyme, since the AJ60 recombination signal sequence does not conform to the established consensus motif and begins with a CAT triplet and not with a CAC triplet. There is therefore no creation of an ApaL1 restriction site when the signal junction is formed.

FIGS. 9B to 9E show that, when this analysis is applied to samples of blood leukocytes purified from various donors, the profile obtained is different for each patient, indicating that each patient has a signal junction repertoire which is specific to said patient.

In order to determine the nature of the modifications, the ApaL1-resistant products were purified on a gel and cloned, except for those derived from the recombination of AJ60 which, for the reasons mentioned above, were cloned without prior digestion. Plasmids present in randomly selected colonies were sequenced (FIG. 10).

The presence of N nucleotides was obvious in all the SJ ApaL1-R analyzed and in some of the hδRec-1/AJ60 SJs. On average, 5.35 nucleotides were added per sequence, the number ranging from 1 to 11. In six cases (19%), bases from one of the signal elements were lost. These deletions range from 1 to 13 bases. Here again, only two sequences were found twice, thereby showing the diversity of the repertoire of the modified SJs.

The δRec-1/AJ signal junctions in human thymocytes therefore exhibit junctional diversity.

The inventors subsequently sought to determine whether the rearrangement of the murine δRec-1 element is restricted to the AJ61 pseudogene or whether, as in the human thymus, other AJ genes can also be used. The same approach was used, and the SJs resulting from the recombination of δRec-1 with AJ61, AJ58, AJ57 and AJ56 were amplified from murine thymocyte DNA. These SJs were readily detectable, as shown in FIG. 11. In addition, the inventors detected rearrangements of δRec-1 with other, more distant AJ genes, up to AJ18. These results demonstrate that the SJs involving the murine δRec-1 element exhibit, as in humans, combinatorial diversity. Since the RSS of the murine δRec-1 element contains an ApaL1 site, the junctional diversity of these SJs cannot be tested directly by restriction. The δRec-1/AJ61 SJs were consequently directly cloned and sequenced. Out of 34 SJs analyzed, 11 (31.42%) were modified (FIG. 12). They all contain N nucleotides, from 2 to 12 per sequence, with an average of 4.4 per sequence. In one of the SJs 3 bases had also been deleted from δRec-1. Once again, the absence of redundant sequences illustrates the diversity of the murine repertoire of SJs involving δRec-1.

These results demonstrate that the recombination of the murine δRec-1 element in wild-type thymocytes generates an SJ repertoire which exhibits combinatorial and junctional diversity.

### REFERENCES

Candéias, S., K. Muegge, et al. (1996). “Junctional diversity in signal joints from T cell receptor beta and delta loci via terminal deoxynucleotidyl transferase and exonucleolytic activity.” J Exp Med 184(5): 1919-26.

Douek, D. C., R. D. McFarland, et al. (1998). “Changes in thymic function with age and during the treatment of HIV infection.” Nature 396(6712): 690-5.

Gilfillan, S., A. Dierich, et al. (1993). “Mice lacking TdT: mature animals with an immature lymphocyte repertoire.” Science 261(5125): 1175-8.

Hazenberg, M. D., S. A. Otto, et al. (2000). “Increased cell division but not thymic dysfunction rapidly affects the T-cell receptor excision circle content of the naive T cell population in HIV-1 infection.” Nat Med 6(9): 1036-42.

Hochberg, E. P., A. C. Chillemi, et al. (2001). “Quantitation of T-cell neogenesis in vivo after allogeneic bone marrow transplantation in adults.” Blood 98(4): 1116-21.

Hodges, E., M. T. Krishna, et al. (2003). “Diagnostic role of tests for T cell receptor (TCR) genes.” J Clin Pathol 56(1): 1-11.

Jung, D. and F. W. Alt (2004). “Unraveling V(D)J recombination; insights into gene regulation.” Cell 116(2): 299-311.

Kanari, Y., R. Nakagawa, et al. (1998). “Variable gene segment-specific N-insertions at the signal joint of T-cell receptor Vbeta-Dbeta recombinations.” Immunol Lett 61(2-3): 151-5.

Schönland, S. O., J. K. Zimmer, et al. (2003). “Homeostatic control of T-cell generation in neonates.” Blood 102(4): 1428-34.

Shutter, J., J. A. Cain, et al. (1995). “A delta T-cell receptor deleting element transgenic reporter construct is rearranged in alpha beta but not gamma delta T-cell lineages.” Mol Cell Biol 15(12): 7022-31.

1. A method of determining the diversity of T lymphocytes in a biological sample from a human patient or from an animal, comprising analysis of the combinatorial diversity and/or the junctional diversity of the excision circles (TRECs) resulting from the excision of all or part of the TCRD locus.

2. The method as claimed in claim 1, comprising analysis of the combinatorial diversity and/or the junctional diversity of the excision circles (TRECs) resulting from the rearrangement of δRec-1.

3. The method as claimed in claim 2, wherein the combinatorial diversity of the excision circles resulting from the rearrangement of δRec-1 is analyzed by carrying out at least three amplification reactions on a fragment of the excision circles, each amplification reaction being specific for a signal junction resulting from the rearrangement of δRec-1 with an AJ region chosen from the group consisting of AJ61, AJ60, AJ59, AJ58, AJ57 and AJ56.

4. The method as claimed in claim 3, wherein the nucleic acid amplification reactions are polymerase chain reactions (PCRs), carried out with pairs of primers each consisting of a primer specific for δRec-1 and of a primer specific for an AJ region chosen from the group consisting of AJ61, AJ60, AJ59, AJ58, AJ57 and AJ56.

5. The method as claimed in claim 3, wherein three or four amplification reactions are carried out, each amplification reaction being specific for a signal junction resulting from the rearrangement of δRec-1 with an AJ region chosen from the group consisting of AJ61, AJ58, AJ57 and AJ56.

6. The method as claimed in claim 5, comprising a step of analysis of the diversity of the signal junctions present on the excision circles resulting from the rearrangement of δRec-1 with at least one AJ region chosen from the group consisting of AJ61, AJ60, AJ59, AJ58, AJ57 and AJ56.

7. The method as claimed in claim 6, wherein each δRec-1/AJ signal junction analyzed, the analysis of the junctional diversity comprises a step of amplification of a fragment of the excision circle, said fragment comprising said signal junction.

8. The method as claimed in claim 7, wherein each δRec-1/AJ signal junction analyzed and resulting from the rearrangement of δRec-1 with an AJ region chosen from the group consisting of AJ61, AJ59, AJ58, AJ57 and AJ56 in humans or AJ61, AJ58, AJ57 and AJ56 in mice, the analysis of the junctional diversity also comprises a step of analyzing the restriction profile, with the ApaL1 enzyme, of the fragment of the excision circle amplified.

9. The method as claimed in claim 6, comprising qualitatively analyzing the signal junctions present on the excision circles resulting from the rearrangement of δRec-1 with at least one AJ region chosen from the group consisting of AJ61, AJ60, AJ59, AJ58, AJ57 and AJ56.

10. The method as claimed in claim 8, further comprising qualitatively analyzing the signal junctions partially or completely resistant to the restriction with ApaL1.

11. The method as claimed in claim 9, wherein the qualitative analysis of the signal junctions is carried out by labeled primer extension.

12. The method as claimed in claim 1, comprising the following steps:
a. preparing the DNA from the biological sample;
b. carrying out at least 3 or 4 PCRs, using pairs of primers, each of which consists of a primer specific for δRec-1 and a primer specific for an AJ region chosen from the group consisting of AJ61, AJ58, AJ57 and AJ56;
c. digesting the PCR products with the ApaL1 restriction enzyme;
d. analyzing the restriction profiles obtained in step c;
e. where appropriate, qualitatively analyzing the signal junctions resistant to the ApaL1 restriction.

13. The method as claimed in claim 1, comprising a step of quantifying the excision circles resulting from the rearrangement of δRec-1 with at least one AJ region chosen from the group consisting of AJ61, AJ60, AJ59, AJ58, AJ57 and AJ56.

14. The method as claimed in claim 1, wherein the method is suitable for detecting a possible disorder of the mechanism of DNA repair.

15. A kit of reagents for determining the diversity of the T lymphocytes present in a biological sample, characterized in that it comprises a collection of at least four primers, at least one of which is specific for δRec-1, and at least three of which are each specific for a different AJ region, selected from the group consisting of AJ61, AJ60, AJ59, AJ58, AJ57 and AJ56, said primers being chosen in such a way as to allow the PCR amplification of the signal junctions resulting from the rearrangement of δRec-1 with the selected AJ regions.

16. The kit of reagents as claimed in claim 15, comprising a collection of at least five primers, at least one of which is specific for δRec-1, at least one of which is specific for AJ61, and at least three of which are each specific for a different AJ region, selected from the group consisting of AJ60, AJ59, AJ58, AJ57 and AJ56, said primers being chosen in such a way as to allow the PCR amplification of the signal junctions resulting from the rearrangement of δRec-1 with the selected AJ regions.

17. The method as claimed in claim 4, wherein the primers are selected from the primers of sequences SEQ ID NO. 1 to 28.

18. The method as claimed in claim 12 wherein the primers are selected from the primers of sequences SEQ ID NO: 1 to 28.

19. The kit as claimed in claim 15 wherein the primers are selected from the primers of sequences SEQ ID NO: 1 to 28.
